(19) United States
(12) Patent Application Publication
(10) Pub. No.: US 2005/0209315 A1
(43) Pub. Date: Sep. 22, 2005

(54) Bioavailable nutritional supplement and method of treatment of malabsorption

(76) Inventors: Andreas Michael Papas, Kingsport, TN (US); Konstantinos Andreas Papas, Jonesborough, TN (US)

Correspondence Address: Donna J. Russell, 1492 Anthony Way, Mt. Juliet, TN, 37122, US

(21) Appl. No.: 11/038,618

(22) Filed: Jan. 19, 2005

Related U.S. Application Data
(63) Continuation-in-part of application No. 10/805,122, filed on Mar. 20, 2004.

(51) Int. Cl. A61K 31/355

(52) U.S. Cl. 514/458

(57) ABSTRACT

The invention relates to novel vitamin compositions having increased absorption and bioavailability. Vitamin E compositions are provided, for example, having biologically significant amounts of one or more vitamin E homologues in an aqueous formulation. Also provided is a method for preparing aqueous formulations of lipophilic nutrients.

### CROSS REFERENCE TO RELATED APPLICATIONS

This application is a continuation-in-part of, and claims the benefit of, U.S. patent application Ser. No. 10/805,122, filed Mar. 20, 2004, the disclosure of which is incorporated herein by reference.

### FIELD OF THE INVENTION

The present invention relates to compositions and methods for providing nutrients having increased bioavailability. More particularly, the invention relates to vitamin formulations that increase vitamin absorption and bioavailability.

### BACKGROUND OF THE INVENTION

The absorption and bioavailability of fat-soluble vitamins such as vitamin E varies greatly in healthy individuals. In patients with malabsorption-associated conditions such as cholestasis, cystic fibrosis, inflammatory bowel disease, hepatitis, short bowel syndrome, bariatric surgery, acquired immunodeficiency syndrome (AIDS), and pancreatitis, nutrient absorption may be significantly decreased, resulting in nutrient deficiencies. Premature and low birth weight infants, especially those who develop necrotizing enterocolitis, also experience significantly decreased Vitamin E absorption.

Decreased plasma and tissue concentration of important fat-soluble vitamins can result in deficiency states that cause neurological, hematological and immune complications and may result in serious morbidity and mortality in affected patients. Fat-soluble vitamin deficiencies may also increase oxidative stress in tissues, weakening the immune system and increasing cancer risk.

Vitamin E is composed of eight different homologues: alpha-tocopherol, beta-tocopherol, gamma-tocopherol, delta-tocopherol, alpha-tocotrienol, beta-tocotrienol, gamma-tocotrienol, and delta-tocotrienol. Studies have demonstrated important effects of these various homologues of vitamin E. Unfortunately, the common commercial sources of natural vitamin E (soy, corn, cottonseed, canola, and sunflower oil distillates) contain little or no tocotrienols, and synthetic vitamin E contains alpha-tocopherol without the other tocopherols and tocotrienols.

Alpha-tocopherol is generally provided as an oil-based product. Water soluble forms are available, however, and research has shown that water-solublized lipophilic compounds are more readily absorbed by the gastrointestinal tract. Vitamin E TPGS (TPGS), for example, is used in commercial products including TwinLabs® Liqui-Ee (Twin Laboratories, Inc., Ronkonkoma, N.Y.) as a water soluble form of alpha-tocopherol. TPGS has been shown to increase alpha-tocopherol levels in cholestatic children that were not affected by large doses of an oil-based alpha-tocopherol. (Sokol, R J et al, Gastroenterology, 1987. 93(5): 975-85.) Unfortunately, TPGS has physical properties that often complicate the process of creating stable aqueous formulations. TPGS forms a wax, gel or other non-flowing solid when mixed with different concentrations of water.

Large doses of dietary alpha-tocopherol displace gamma-tocopherol and other homologues in plasma and other tissues. Research has shown that oral ingestion of supplements containing alpha-tocopherol alone depletes gamma-tocopherol levels in blood and tissues. This is proposed to be due to the selective action of alpha-tocopherol-binding protein (α-TTP) in the liver. (Kaempf-Rotzoll D., et al., Curr. Opin. Lipidol. 2003;14:249-254).

The tocopherols differ from one another by the position of the methyl groups on the chromanol ring. Gamma-tocopherol scavenges nitrogen radicals more effectively than alpha-tocopherol. (Wolf G., Nutr. Rev., 1997. 55(10): 376-8.) In vivo experiments with rats have indicated that γ-tocopherol is more effective than α-tocopherol in inhibiting low-density lipoprotein (LDL) oxidation. A metabolite of gamma-tocopherol, 2,7,8-trimethyl-2-(gamma-carboxyethyl)-6-hydroxychroman (gamma-CEHC), has natriuretic activity (Wechter W. J. et al, Proc. Natl. Acad. Sci. USA (1996) 93: 6002-6007). Gamma-tocopherol has been shown to reduce PGE2 synthesis and is being investigated in inflammatory disorders. In addition, gamma-tocopherol is being investigated for prostate cancer therapy, as well as for other disease therapies (Helzlsouer K J, et al, J. Natl. Cancer Inst. 2000;92:2018-2023).

Plasma vitamin E levels (which are almost exclusively alpha-tocopherol, when measured) have shown a strong inverse correlation with coronary heart disease. When dietary sources were compared, however, studies indicated that supplements containing alpha-tocopherol showed no protective effect, while consumption of natural dietary sources of vitamin E (magarine, nuts, seeds) containing substantial amounts of gamma-tocopherol, did produce a demonstrable protective effect (Kushi, L., et al., N. Engl. J. Med. (1996) 334: 1156-1162). In individuals suffering from coronary heart disease, serum levels of gamma-tocopherol were shown to be decreased as compared to normal, but alpha-tocopherol levels were not (Ohrvall, M., et al., J. Intern. Med. (1996) 239: 111-117).

Gamma-tocopherol removes peroxynitrite-derived species to protect against peroxynitrate-induced lipid peroxidation (Christen, S., et al., Proc. Natl. Acad. Sci. USA (1997) 94: 3217-3222). Gamma-tocopherol has stronger anti-inflammatory properties than alpha-tocopherol, reducing PGE2 synthesis in both macrophages and human epithelial cells, while alpha-tocopherol slightly reduces PGE2 formation in macrophages but has demonstrated no effect in epithelial cells.

Tocotrienols have an unsaturated isoprenoid side chain (as opposed to the unsaturated phytyl side chain of tocopherols), allowing them to penetrate the cell membrane more easily. Alpha tocotrienol has 40-60% more antioxidant activity than alpha tocopherol. Tocotrienols have been shown to reduce cholesterol in human clinical trials by increasing the conversion of farnesyl to farnesol, which is a post-transcriptional inhibitor of HMG Co-A reductase. (Theriault, A. et al., Clin. Biochem., (1999) 32(5): 309-19.) Tocotrienols have also been shown to reduce carotid stenosis in clinical studies (Tomeo, A. et al., Lipids (1995) 30(12): 1179-83). Tocotrienols have been shown to inhibit breast cancer cell growth in vitro and to decrease glutamate-induced death of neuronal cells (Takahashi K, Loo G, Biochem. Pharmacol. (2004) 67(2): 315-24; Sen, C. et al. J. Biol. Chem. (2000) 275(17): 13049-55).

Although the primary purpose of nutritional supplements is to provide desirable vitamins, minerals, and other nutritional components that are not contained in the diet or are not adequately absorbed from the diet, many current preparations contain only portions of the necessary elements. As is the case with vitamin E, some of those components may be more readily absorbed, some may be selectively transported, and the relative amounts of some may have an inverse effect on the effective amounts of others in certain body tissues. What is needed are formulations that provide more complete nutritional benefit by providing multiple vitamin homologues in a more absorbable and bioavailable form.

### SUMMARY OF THE INVENTION

The present invention provides a composition comprising an aqueous emulsion comprising a therapeutically effective amount of at least one Vitamin E homologue (VEH) and an effective amount of d-α-tocopheryl polyethylene glycol 1000 succinate (Vitamin E TPGS), alone or in conjunction with a co-emulsifier, to solubilize the VEH in the aqueous phase. In its various embodiments, the composition of the invention can comprise vitamin E homologue is selected from the group consisting of alpha-tocopherol, beta-tocopherol, gamma-tocopherol, delta-tocopherol, alpha-tocotrienol, beta-tocotrienol, gamma-tocotrienol, delta-tocotrienol, and combinations thereof.

In one embodiment of the invention, the composition comprises an emulsion wherein the aqueous phase comprises about 80 to about 99 weight percent and the lipid phase comprises about 1 to about 20 weight percent. In certain embodiments, the lipid phase comprising the lipophilic vitamin E homologues and TPGS can also comprise at least one lipophile chosen from the group comprising coenzyme Q10, carotenoids, alpha-lipoic acid, essential fatty acids, and combinations thereof.

Embodiments of the invention include compositions Wherein the at least one Vitamin E homologue comprises about 25 to about 50 weight percent alpha-tocopherol; about 0.1 to about 5 weight percent beta-tocopherol; about 25 to about 50 weight percent gamma-tocopherol; about 5 to about 25 weight percent delta-tocopherol; about 0.1 to about 5 weight percent alpha-tocotrienol; about 0.1 to about 5 weight percent beta-tocotrienol; about 0.1 to about 5 weight percent gamma-tocotrienol; about 0.1 to about 5 weight percent delta-tocotrienol; and combinations thereof.

The invention also provides a method of making a composition comprising at least one VEH according to the present invention. The method for making compositions comprising stable emulsions of one or more vitamin E homologues can comprise the steps of heating a mixture of lipids comprising about 10 to about 75 weight percent of at least one vitamin E homologue, about 10 to about 75 weight percent Vitamin E TPGS alone or in conjunction with a co-emulsifier, and a lipid sufficient to provide a total of 100 weight percent, thereby producing a lipid phase; combining the lipid phase with an amount of water sufficient to equal about 80 to about 99 weight percent water; and admixing the lipid phase and water for a period of from about 2 to about 8 hours at temperature of about 45 to about 55 degrees C. to provide an emulsion that is stable at room temperature.

A method for increasing absorption of one or more VEHs and for treating a mammalian subject with malabsorption resulting from a disease or other condition is also provided, the method comprising administering to a subject an effective amount of a composition described by the present invention.

### DETAILED DESCRIPTION

The present invention relates to a stable water-soluble formulation that consists of an emulsion of multiple homologues of vitamin E (VEHs). The inventors have developed a composition comprising an aqueous emulsion having a therapeutically effective amount of at least one VEH and a concentration of Vitamin E TPGS, alone or in conjunction with a co-emulsifier, that is effective for solubilizing the VEH in the aqueous phase.

Vitamin ETPGS(TPGS) is a water-soluble form of natural-source vitamin E prepared by esterifying d-α-tocopheryl acid succinate with polyethylene glycol 1000 to produce d-α-tocopheryl polyethylene glycol-1000-succinate. It generally has the chemical formula of C₃₃O₅H₅₄(CH₂ CH₂O)ₙ, where “n” represents the number of polyethylene oxide moieties attached to the acid group of crystalline d-alpha tocopheryl acid succinate.

In the composition provided by the inventors, the aqueous emulsion comprises lipophilic VEH dispersed throughout an aqueous phase. The emulsion comprises a blend of a therapeutically effective amount or concentration of at least one VEH and a concentration of TPGS, alone or in conjunction with a co-emulsifier, so that the concentration of TPGS alone or in conjunction with co-emulsifier is effective for solubilizing the lipophile in an aqueous phase, such as water.

Although TPGS has been described as an emulsifier, its ability to emulsify certain lipophiles is unpredictable, and its behavior when admixed with water can make it less than ideal for preparing formulations in which it provides for emulsification of other ingredients. The inventors have discovered effective ratios of VEH to TPGS to an aqueous component, such as water, that can be used to create stable aqueous emulsions to provide more readily absorbed vitamin preparations. An aqueous emulsion using TPGS and multiple homologues of vitamin E (alpha-tocopherol, beta-tocopherol, gamma-tocopherol, delta-tocopherol, alpha-tocotrienol, beta-tocotrienol, gamma-tocotrienol, delta-tocotrienol, and combinations thereof) can be formulated by combining appropriate ratios of TPGS, VEH and water using the method provided by the present invention. In such compositions, the aqueous phase comprises about 80 to about 99 weight percent and lipid phase comprises about 1 to about 20 weight percent. The VEH weight percent can be reduced when a co-emulsifier is used.

A VEH composition comprises, for example: 1) about 25 to about 50 weight percent alpha-tocopherol; 2) about 0.1 to about 5 weight percent beta-tocopherol; 3) about 25 to about 50 weight percent gamma-tocopherol; 4) about 5 to about 25 weight percent delta-tocopherol; 5) about 0.1 to about 5 weight percent alpha-tocotrienol; 6) about 0.1 to about 5 weight percent beta-tocotrienol; 7) about 0.1 to about 5 weight percent gamma-tocotrienol; and 8) about 0.1 to about 5 weight percent delta-tocotrienol. It is to be understood that the invention may also include only a portion of these vitamin E homologues. The composition may also comprise additional ingredients such as, for example, other lipophilic nutrients, excipients, stabilizers, and preservatives such as, for example, potassium sorbate, sorbic acid, benzoates and their sodium, potassium, and calcium salts, sulphites, acetate, propionate, and citrates.

In order to provide efficient utilization of VEH, it is preferable to provide a ratio of alpha tocopherol as IU to the sum of the other homologues that is about 1:1. A preferred range for the alpha tocopherol NEH ratios is about 0.67 to about 1.33, and the effective ratio range is about 0.25 to about 1.75. Preferably, the alpha tocopherol (measured as IU) to gamma tocopherol ratio is approximately 1.5, or within a range of about 1.0 to about 1.75, with an effective ratio range of about 0.4 to about 1.90. A range of preferred ratios of tocopherols to tocotrienols is 1.0 to 1.75, with an effective ratio of 0.4 to 1.90.

Compositions provided by the present invention may be provided in liquid form, in capsules, softgels or other coatings, and by other means known to those of skill in the pharmaceutical arts. Single dose units or multiple dose units, such as bottles or vials from which single dose amounts may be readily obtained, for example, may be provided. Compositions may also be provided in dose units to be administered with or without food, or may be incorporated into food formulations, such as beverages or infant formulas. Compositions may be provided by incorporating the aqueous emulsion into a food or beverage, or by coating the surface of the food with the emulsion, such as by spray coating a wafer, cookie, or other food.

Compositions may also be provided for administration to non-human subjects, such as, for example, canine or feline mammals. VEH compositions of the present invention can be provided for veterinary use in vials, capsules, or other formulations for administration to an animal as a supplement with or without concurrent ingestion of food, or may be provided as a liquid that can be spray-coated on foods, or incorporated into or sprayed or otherwise distributed onto the surface of a moist or dry food.

Compositions provided by the invention may provide, for example, daily doses of approximately 1.5 ml to supply 30 IU for children, plus about 27 mg of the non-alpha homologues (including approximately 18 milligrams of gamma tocopherol and approximately 1.8 milligrams of tocotrienols). Doses may be adjusted by caregivers to provide amounts appropriate for newborn babies or children with specific nutritional needs, for example.

Compositions provided by the invention may provide, for example, daily doses of 100 IU in 5 ml plus 90 mg of the non-alpha homologues (including 60 mg of gamma tocopherol and about 9 mg of tocotrienols). Dose may be adjusted by those of skill in the art to provide lower amounts or higher amounts, as needed. Individuals with significant malabsorption or special nutritional or medical conditions may be provided with higher doses, for example.

The invention also provides a method for formulating stable VEH emulsions. The method comprises producing a lipid phase by heating and blending a mixture of lipids comprising about 10 to about 75 weight percent of a therapeutically effective lipophile, about 10 to about 75 weight percent Vitamin E TPGS alone or in conjunction with a co-emulsifier, and a lipid sufficient to provide a total of 100 weight percent after the lipophile, TPGS, and any other desired ingredients are taken into account. The lipid phase is contacted with an amount of water to form an about 80 to about 99 weight percent aqueous mixture, and the emulsion is admixed for a period of from about 2 to about 8 hours, more preferably from about 4 to about 6 hours, at temperature of about 45 to about 55 degrees C., or about 48 to about 52 degrees C., to provide an emulsion that is stable at room temperature and has a particle size that facilitates increased absorption or increased bioavailability. In one embodiment, for example, compositions are mixed and heated for about 5 hours at approximately 50° C.

The invention also provides a method for increasing nutrient absorption in a subject experiencing malabsorption of that nutrient, which can often occur in certain disease states such as, for example, cholestasis, cystic fibrosis, inflammatory bowel disease, hepatitis, short bowel syndrome, bariatric surgery, AIDS, and pancreatitis. An aqueous emulsion as described by the invention has been shown in clinical trials to increase the absorption of the vitamin E homologues, especially the non-alpha-tocopherol homologues, in patients with malabsorption syndromes.

Compositions provided by the invention can be provided to infants, toddlers, children, or adults. A composition comprising Vitamin E homologues can, for example, be provided to an infant as a nutritional supplement or as a component of an infant formula to increase the effective amounts of those homologues. This may be especially beneficial in infants who fail to thrive, suffer from a malabsorption syndrome, or have a condition such as necrotizing enterocolitis that decreases nutrient absorption.

Compositions provided by the invention may be provided to non-human subjects, as well. Veterinary applications of the compositions and method of the invention can include, for example, administration to puppies in milk substitutes or early foods. Administration as a supplement or as a component of a pet food such as kibble, particularly when a VEH composition is spray-coated or otherwise applied to the surface of the kibble, can provide a health benefit to both healthy animals and to animals with nutritional deficit due to malabsorption or disease, as well as animals in which Vitamin E therapy may be especially therapeutic-such as in dogs with chronic hepatitis, a condition for which Vitamin E therapy is often used.

The invention may be further described by means of the following non-limiting examples.

### EXAMPLE 1

Preparation of a VEH Composition

Amounts of Vitamin E homologues in a VEH composition prepared as described by the method of the present invention are shown in Table 1.

TABLE 1

| Tocopherol and Tocotrienol | Amount | % |
|---|---|---|
| in Aqueous Formulation | per 10 ml | Daily Value |
| IU from d-alpha-tocopherol | 200 | 667% |
| d-gamma-Tocopherol, mg | 117 | * |
| d-beta + d-delta-Tocopherol, mg | 42 | * |
| Total tocopherols, mg | 296 | * |
| Total tocotrienols, mg | 17 | * |
| Total tocopherols + tocotrienols, mg | 313 | * |

Briefly, the indicated amounts of the vitamin E homologues (or other lipophilic components) were weighed, added to a Hamilton Kettle, and admixed. The mixture was then heated to approximately 49-51° C. Approximately ⅓ (90,000 g) of the total amount (300 liters) of purified water and potassium sorbate (to give 0.125% by weight) were weighed, added to a Groen Kettle and heated to 80° C. The potassium sorbate/water mixture was added to the lipophilic ingredients in the Hamilton Kettle and the combination was mixed at high speed to produce a vortex while avoiding air entrapment. The remaining approximately ⅔ (180,000 g) of total purified water was placed in the Groen Kettle and heated to 80° C., then transferred to the Hamilton Kettle. The contents of the Hamilton Kettle were then mixed with a lighting mixer until the mixture in Hamilton Kettle reached 23-25° C. Purified water was then added to reach the desired batch weight or volume.

### EXAMPLE 2

Two patients with documented cystic fibrosis and malabsorption, requiring the use of pancreatic enzymes and supplemental vitamin E, were randomized to a single dose of either a typical oil-based softgel formulation or the water-soluble formulation described by the inventors following a washout period of three weeks in which all supplemental vitamin E was discontinued.

Three softgels and 20 ml of the water-soluble formulation contained the same amount of gamma-tocopherol, as well as the other tocopherols and tocotrienols. Plasma measurements were taken at time 0, 2, 4, 8, 24, 48 and 168 hours. The data in Table 2 are the measured plasma levels of gamma-tocopherol at each time point. As these numbers indicate, the aqueous formulation has a bioavailability of almost twice that of the oil-based preparation.

TABLE 2
Gamma-tocopherol plasma concentrations (mcg/ml)

| Time | Patient 1 | Patient 2 |
|---|---|---|
| (Hours) | (water-soluble formulation) | (oil-based formulation) |
| 0 | 0.645 | 0.614 |
| 2 | 0.699 | 0.940 |
| 4 | 0.822 | 1.97 |
| 8 | 1.44 | 1.87 |
| 24 | 2.05 | 1.4 |
| 48 | 1.22 | 0.818 |
| 168 | 0.827 | 0.596 |

1. An aqueous emulsion comprising an aqueous phase and a lipid phase, the lipid phase comprising
a) at least one vitamin E homologue,
b) an effective amount of Vitamin E TPGS to solubilize the vitamin E homologue in the aqueous phase.

2. An aqueous emulsion as in claim 1 wherein the vitamin E homologue is selected from the group consisting of alpha-tocopherol, beta-tocopherol, gamma-tocopherol, delta-tocopherol, alpha-tocotrienol, beta-tocotrienol, gamma-tocotrienol, delta-tocotrienol, and combinations thereof.

3. An aqueous emulsion as in claim 2 wherein the ratio of alpha-tocopherol to the sum of the other vitamin E homologues is about 0.67 to about 1.33.

4. An aqueous emulsion as in claim 3 wherein the ratio of alpha-tocopherol to the sum of the other vitamin E homologues is about 1.

5. An aqueous emulsion as in claim 2 wherein the ratio of alpha-tocopherol to gamma-tocopherol is about 1.0 to about 1.75.

6. An aqueous emulsion as in claim 5 wherein the ratio of alpha-tocopherol to gamma-tocopherol is about 1.5.

7. The emulsion of claim 1 wherein the aqueous phase comprises about 80 to about 99 weight percent and the lipid phase comprises about 1 to about 20 weight percent.

8. An emulsion according to claim 1 wherein the lipid phase further comprises at least one lipophile chosen from the group comprising coenzyme Q10, carotenoids, alpha-lipoic acid, essential fatty acids, and combinations thereof.

9. The emulsion according to claim 1 wherein the at least one Vitamin E homologue comprises
a) about 25 to about 50 weight percent alpha-tocopherol;
b) about 0.1 to about 5 weight percent beta-tocopherol;
c) about 25 to about 50 weight percent gamma-tocopherol;
d) about 5 to about 25 weight percent delta-tocopherol;
e) about 0.1 to about 5 weight percent alpha-tocotrienol;
f) about 0.1 to about 5 weight percent beta-tocotrienol;
g) about 0.1 to about 5 weight percent gamma-tocotrienol;
h) about 0.1 to about 5 weight percent delta-tocotrienol;
or a combination thereof.

10. The emulsion according to claim 9 wherein the at least one Vitamin E homologue comprises
a) about 25 to about 50 weight percent alpha-tocopherol;
b) about 0.1 to about 5 weight percent beta-tocopherol;
c) about 25 to about 50 weight percent gamma-tocopherol;
d) about 5 to about 25 weight percent delta-tocopherol;
e) about 0.1 to about 5 weight percent alpha-tocotrienol;
f) about 0.1 to about 5 weight percent beta-tocotrienol;
g) about 0.1 to about 5 weight percent gamma-tocotrienol; and
h) about 0.1 to about 5 weight percent delta-tocotrienol.

11. A method for formulating stable emulsions of one or more vitamin E homologues, the method comprising
a) heating a mixture of lipids comprising about 10 to about 75 weight percent of a at least one vitamin E homologue, about 10 to about 75 weight percent Vitamin E TPGS alone or in conjunction with a co-emulsifier, and a lipid sufficient to provide a total of 100 weight percent, thereby producing a lipid phase;
b) combining the lipid phase with an amount of water sufficient to equal about 80 to about 99 weight percent water;
c) admixing the lipid phase and water for a period of from about 2 to about 8 hours at temperature of about 45 to about 55 degrees C. to provide an emulsion that is stable at room temperature.

12. The method of claim 11 wherein the emulsion provides Vitamin E homologues having a particle size that facilitates increased absorption or increased bioavailability in mammalian tissue.

13. The method of claim 11 wherein the step of admixing the lipid phase and water comprises a period of from about 4 to about 6 hours.

14. The method of claim 11 wherein the step of admixing the lipid phase and water comprises a period of about 5 hours.

15. The method of claim 11 wherein the step of admixing the lipid phase and water comprises admixing at a temperature of about 48 to about 52 degrees C.

16. The method of claim 11 wherein the step of admixing the lipid phase and water comprises admixing at a temperature of about 50 degrees C.
